(19) United States
(12) Patent Application Publication
(10) Pub. No.: US 2010/0191136 A1
(43) Pub. Date: Jul. 29, 2010

(54) SYSTEM, PAD AND METHOD FOR MONITORING A SLEEPING PERSON TO DETECT AN APNEA STATE CONDITION

(76) Inventor: Danette K. Wolford, Westminster, MD (US)

Correspondence Address: CHARTER IP, LLC, P.O. BOX 64, The Plains, VA, 20198, US

(21) Appl. No.: 12/359,459

(22) Filed: Jan. 26, 2009

(51) Int. Cl. A61B 5/08; A61H 1/00

(52) U.S. Cl. 600/534; 601/46

(57) ABSTRACT

A system, pad and method of monitoring a sleeping person to detect an apnea state condition enables the person to be subject to a tactile action. In the system, a pad is provided beneath the person, and a plurality of independent position sensors are arranged within the pad for measuring movement of the person at different locations. A main control unit in electrical communication with the position sensors, and a plurality of independent vibration devices are arranged within the pad and in electrical communication with the main control unit. The main control unit is configured to receive signals from the position sensors indicative of a stoppage of breathing in the person for a given duration, and to transmit control signals to cause the vibration devices to generate a tactile action in the pad so as to jog the person out of the apnea state.

### BACKGROUND

1. Field of the Invention

Example embodiments in general are directed to a system, pad and method for monitoring a sleeping person to detect an apnea state condition.

2. Description of Related Art

Apnea of infancy may be understood as an unexplained episode of cessation of breathing for 20 seconds or longer, or a shorter respiratory pause associated with bradycardia, cyanosis, pallor, and/or marked hypotonia. The term “apnea of infancy” generally refers to infants with gestational age of 37 weeks or more at the onset of apnea.

Apnea of prematurity may be understood as the sudden cessation of breathing that lasts for at least 20 seconds or is accompanied by bradycardia or oxygen desaturation (cyanosis) in an infant younger than 37 weeks' gestational age. It usually ceases by 37 weeks' postmenstrual age but may persist for several weeks beyond term.

Peer-reviewed evidence among physicians indicates that apnea is not necessarily predictive of or a precursor to Sudden Infant Death Syndrome (SIDS). There appears to be no clear, unequivocal relationship between apnea and SIDS. However, several studies offer evidence that although apnea has not been proven a precursor for SIDS, certain infants who died of SIDS experienced significantly more frequent episodes of obstructive and mixed sleep apnea then other infants included within the same test groups.

Apnea monitors were first introduced in the mid-1960s for the management of apnea of prematurity in hospital settings. Subsequently, cardio-respiratory monitoring has become widely used in the care of infants with a variety of acute and chronic disorders. Within hospitals, premature and newly born infants.

Today, most if not all U.S. hospitals which have a neo-natal intensive care unit (NICU) include equipment that monitors the breathing rate and/or heart rate of infants, among other vitals, so as to detect an apnea state in the infant. Upon detection of the state, an audible alarm may sound to alert the caregiver. Home apnea monitoring systems also typically provide a similar audible warning and/or a warning LED. Neither the NICU nor home systems provide a proactive means of physically alerting the patient or infant.

### SUMMARY

An example embodiment of the present invention is directed to a system for monitoring a sleeping person to detect an apnea state condition. In the system, a pad is provided beneath the person, and a plurality of independent position sensors are arranged within the pad for measuring movement of the person at different locations. The system includes a main control unit in electrical communication with the position sensors, and a plurality of independent vibration devices arranged within the pad and in electrical communication with the main control unit. The main control unit is configured to receive signals from the position sensors indicative of a stoppage of breathing in the person for a given duration, and to transmit control signals to cause the vibration devices to generate a tactile action in the pad so as to jog the person out of the apnea state.

Another example embodiment is directed to a pad for monitoring a sleeping person to detect an apnea state condition. The pad includes a pair of sheets encasing a gel material therein for placement on a mattress or for use as a mattress, a plurality of independent position sensors within the gel material for measuring movement of the person at different locations, and a plurality of independent vibration devices within the gel material, the sensors and vibration devices being in electrical communication with a remote main control unit. In response to the sensors sending signals to the main control unit indicative of a stoppage of breathing in the person for a given duration, the vibration devices receive control signals to generate a tactile action in the pad so as to jog the person out of the apnea state.

Another example embodiment is directed to a method of monitoring a sleeping human being to detect an apnea state condition. In the method, a pad is provided under a prone person prior to a sleep event. The pad includes a plurality of independent position sensors therein for measuring movement of the person at different locations and a plurality of independent stimulation devices therein. The plurality of position sensors and stimulation devices are electrically connected to a controller. The sensors monitor movement of the person on the pad so as to track breathing. The controller transmits control signals to cause the stimulation devices to generate a tactile action in the pad so as to jog the person out of the apnea state, if it receives signals from the position sensors indicative of a stoppage of breathing in the person for a given duration.

### BRIEF DESCRIPTION OF THE DRAWINGS

Example embodiments will become more fully understood from the detailed description given herein below and the accompanying drawings, wherein like elements are represented by like reference numerals, which are given by way of illustration only and thus are not limitative of the example embodiments herein.

FIG. 1 is a block diagram of a system for monitoring a sleeping person to detect an apnea state condition in accordance with the example embodiments.

FIG. 2 is a block diagram illustrating component details of the main control unit 130 in more detail.

FIG. 3 is a block diagram of a wireless system for monitoring a sleeping person to detect an apnea state condition in accordance with the example embodiments.

FIG. 4 is flowchart to illustrate the method for monitoring a sleeping person to detect an apnea state condition.

### DETAILED DESCRIPTION

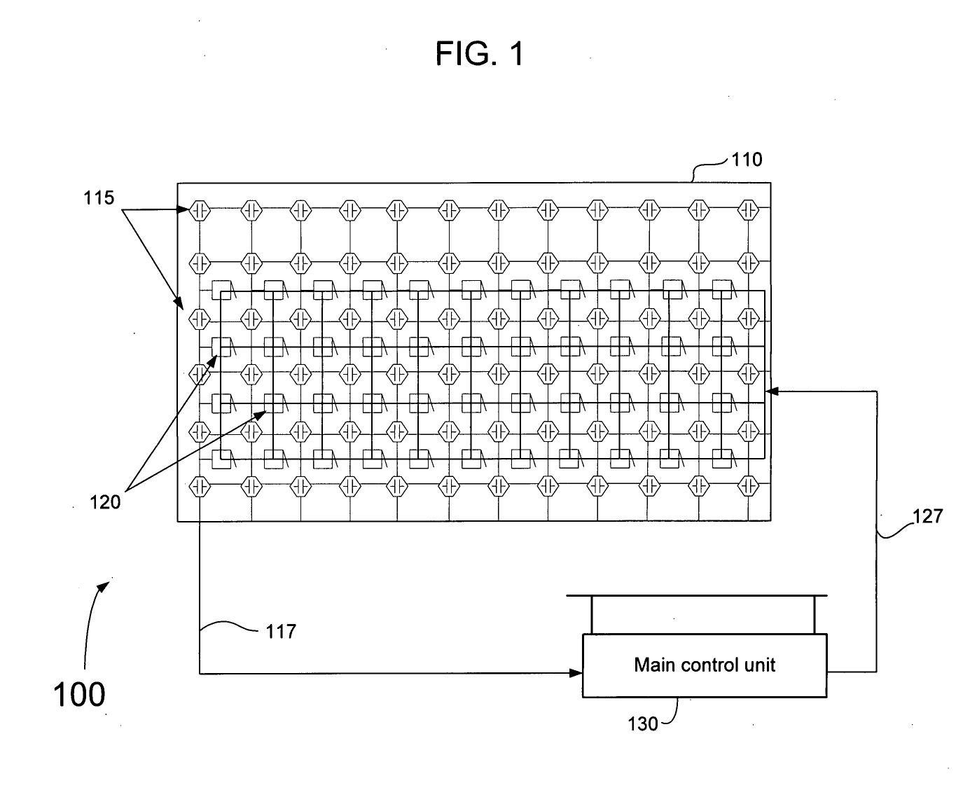

FIG. 1 is a block diagram of a system for monitoring a sleeping person to detect an apnea state condition in accordance with the example embodiments. Referring to FIG. 1, the system 100 may include a pad 110 in communication with a monitoring device or “main control unit 130”. Hereafter the pad shall be referred to as a gel mattress 110. The gel mattress 110 can be used with infants, the elderly, those with respiratory problems and/or sleep apnea patients. The gel mattress 110 is characterized as having a gel substance encased within a thin, polyurethane (or like material) outer layer or encased within a pair of poly sheets attached to each other along a periphery thereof to form the gel mattress 110. The gel mattress 110 contains a number of independent, spaced position sensors 115 therein for measuring movement of the person at different locations sensors, on the order of hundreds for example. The vast numbers of sensors 115 within the gel mattress 110 provide redundancy, in the case where one or multiple sensors 115 fail. The gel mattress 110 may be placed on top of an existing mattress within a crib or bed under the patient, or may serve as a mattress itself.

Using a preemie infant as an example, the sensors 115 should have a sensitivity to detect the stoppage of breathing/movement for as little as a 500 gram weight, equivalent of a 24-week premature infant. As all sensors 115 are contained within the gel mattress 110, no sensors 115 have to be attached to the infant, such as by an adhesive. The sensors 115 in one example can be smart sensors (with a microprocessor chip therein). In this example, the sensors 115 can be programmed to sense one or more of body temperature, heart rate, respiratory rate and breathing/patient movement (or lack thereof). As the sensors 115 are sensitive to movement in as small as a 500 g person (such as a 24 week-old infant), the sensors 115 are sufficiently sensitive for all patients.

Example sensors 115 may include ultra-sensitive movement sensors such as piezoelectric, fiber optic and/or load cell based sensors that provide data signals to the main control unit 130. The sensors 115 are sensitive enough to provide, through known signal processing techniques, heart rate and breathing rate, and additional vital statistics such as body temperature and O₂ saturation, for example. In an alternative, each of the sensors 115 may be comprised of one or more sensor films such as PVDF film strips that can generate a signal between conductive film surfaces formed within the gel mattress 110.

The gel mattress 110 includes a number of independent, spaced stimulation devices for imparting a tactile stimulus to the person, based on a control signal received from the main control unit 130. In one example, the stimulation devices may be embodied as vibration devices 120, although other types of stimulation devices may be employed in lieu of or in addition to vibration devices 120.

The vibration devices 120 may number on the order of tens of devices to hundreds, for example. In an example, the vibration devices 120 may be similar to the vibrators with motors that are employed in many hand-held electronic devices such as cell phones and personal digital assistants (PDAs). The vibration device 120 may include a motor with an offset weight attached to a spindle, such as is commonly used for silent ring in a cell phone. The offset weight causes a vibration when the motor is energized by a control signal received from the main control unit 130, which is thus transferred as a tactile action to the person.

FIG. 1 illustrates a wired configuration, where the sensors 115 are in electrical communication with the main control unit 130 via lead 117, and the vibration devices 120 are in electrical communication with the main control unit 130 via lead 127. The main control unit 130 is configured to receive data signals via lead 117 from the position sensors 115. If the data signals are indicative of a stoppage of breathing in the person (i.e., an apnea state), the main control unit 130 sends or transmits control signals to cause the vibration devices 120 to generate a tactile action in the gel mattress 110 so as to jog the person out of the apnea state.

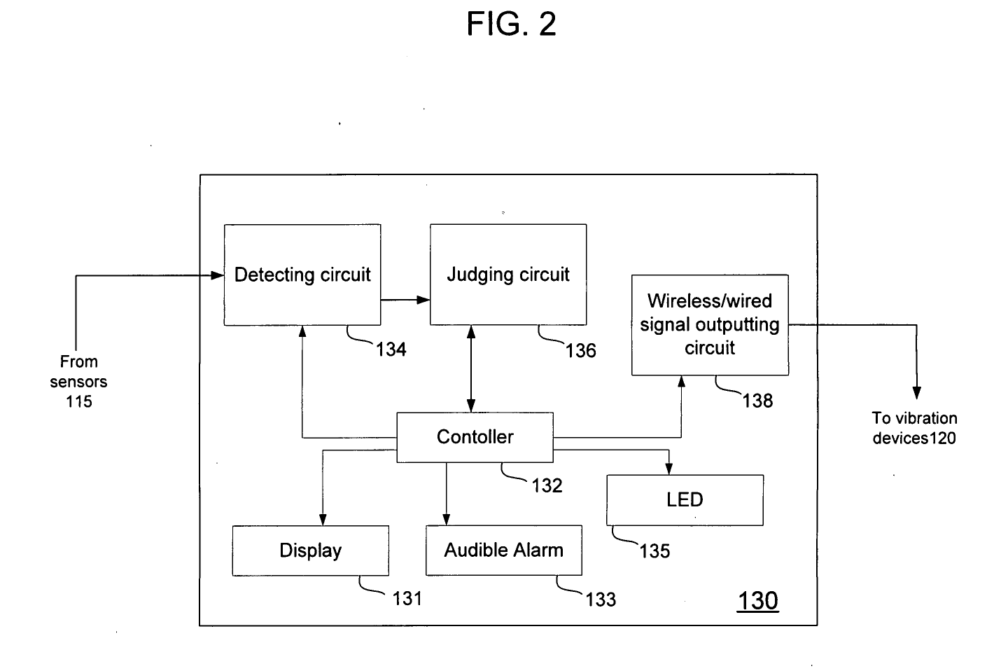

FIG. 2 is a block diagram illustrating component circuits of the main control unit 130 in more detail. Referring to FIG. 2, the main control unit 130 includes a controller (such a microprocessor 132) for implementing and controlling software algorithms designed for detecting the data signals from the sensors 115 (“detecting circuit 134”), judging and comparing the data signals to a given criteria for calculating vital statistics and determining an apnea state (“judging circuit 136”), and for outputting control signals to the vibration devices 120 upon detecting an apnea state (“wireless/wired signal outputting circuit 138”). The specific functions implemented in these algorithms for determining breathing rate, heart rate and/or lack of breathing (apnea state) are well known in the art of patient monitoring systems and are not heretofore described in detail for purposes of brevity.

Additionally, main control unit 130 may include a display 131 to display patient vital information, an optional audible alarm 133 and an optional visual LED alarm 135 that may actuate under control of controller 132 based on a detected apnea state. The main control unit 130 may be powered by AC mains power or by one or more of a replaceable, rechargeable, solar-powered and combination rechargeable and solar-powered battery, for example.

The main control unit 130 and its functionality can be implemented as part of existing neonatal or patient monitoring systems. Example monitoring systems include the Dash® or Solar® monitoring systems by GE Healthcare®, which can be specifically designed for the NICU with CRG trending and configurable alarm management. These monitoring systems include parameters designed to capture vital patient measurements and sophisticated analysis programs to help prevent false arrhythmia alarms.

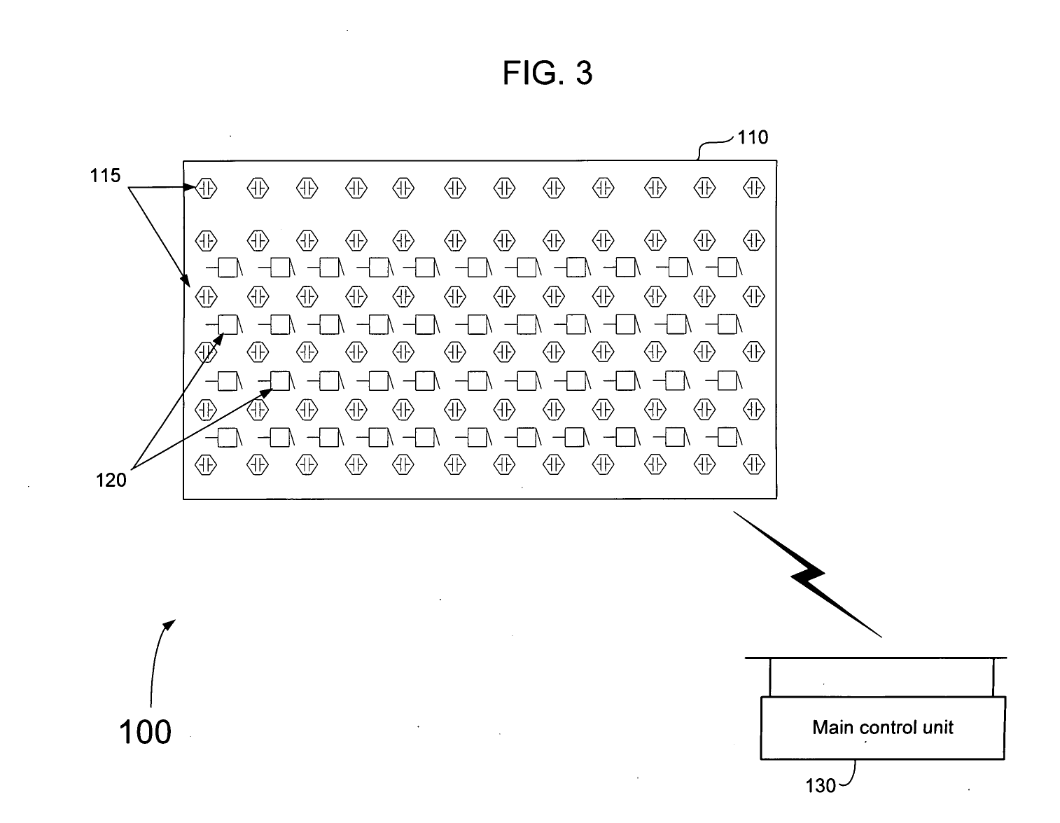

FIG. 3 is a block diagram of a wireless system for monitoring a sleeping person to detect an apnea state condition in accordance with the example embodiments. System 100 can be configured in a wireless embodiment. An example wireless configuration can be a wireless LAN option to permit the main control unit 130 to be moved about the patient room or to other rooms in the NICU or ICU. Using 802.11b technology, the main control unit 130 can be designed to be integrated into an existing wireless network. As shown in FIG. 3, one or more of the gel mattress 110, position sensors 115, vibration devices 120 and main control unit 130 can be configured with tiny transceivers including associated radio and microprocessor components, such that signal communication between components is wireless.

An example electronics software package of the main control unit 130 for neonatal monitoring may be the CRG® 24-hour, beat-to-beat trend package by GE Healthcare®. This software package provides for efficient evaluation of bradycardia and apnea events, as well as dual-pulse oximetry, and transcutaneous monitoring for highly accurate, non-invasive O₂ and CO₂ readings.

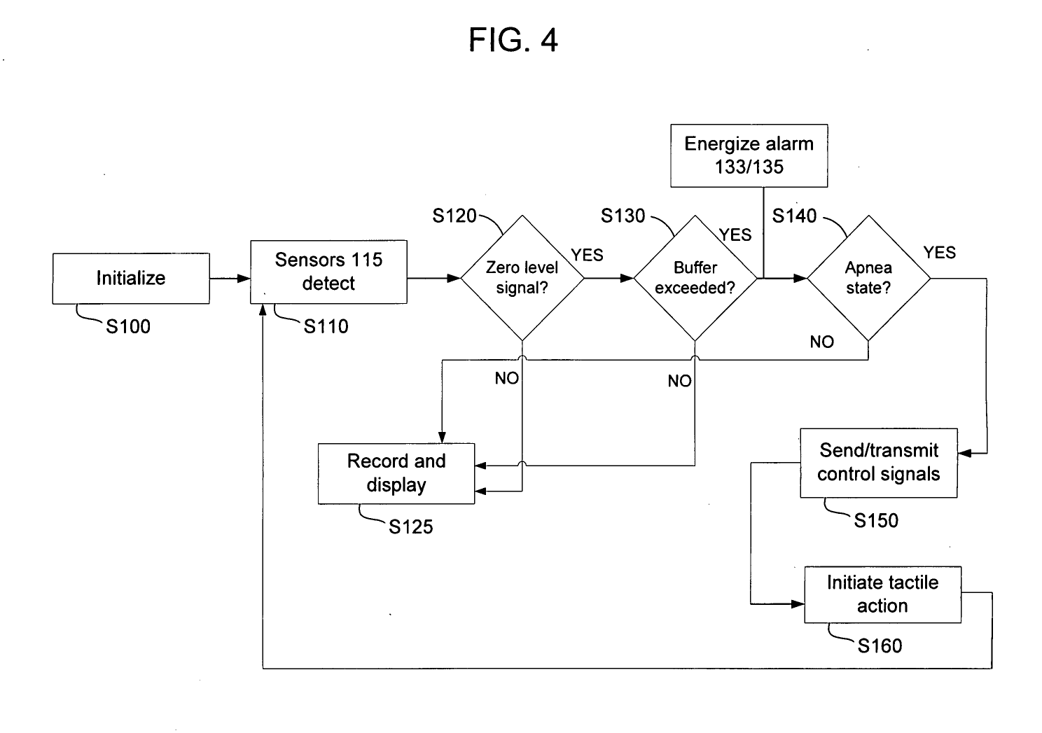

FIG. 4 is flowchart to illustrate a method for monitoring a sleeping person to detect an apnea state condition. Initially, the gel mattress 110 is electrically connected to (or in RF communication with) the main control unit 130. Upon power up, an initiation sequence is performed (S100) to confirm that all sensors 115 and vibration devices 120 are working properly.

In operation, the sensors 115 constantly detect movement of the patient (S110), indicative of breathing, sending regular data signals indicative of movement via lead 117 (or as a wireless signal) to the detecting circuit 134 of the main control unit 130, to be judged and recorded for display on the main control unit 130 (S125). The frequency of sampling by the sensors 115 can be correlated, via software updates to the detection circuit 134, to the frequency at which the patient is breathing. For example, a neonatal or infant typically breathes at a rate of about 60 breaths/minute; a pediatric 20-40 breaths/minute; a teenager/adult about 20 breaths/minute. Accordingly, the sampling rate of the sensors 115 may be adjusted to sample at a rate corresponding to the age and/or weight of the person on gel mattress 110.

In the absence of breathing or movement of the patient, a low level (or zero position movement) data signal is sent (output of S120 is “YES”). To avoid a spurious alarm, a delay or buffer can be inserted, reflected in the control algorithm run by the judging circuit 136 under control of controller 132. The buffer period can be varied depending on the person being monitored; an example range may be between 1.5 to 5 seconds. In another example, a buffer period may be set to 2 seconds for an infant, 4-5 seconds for an adult. If no breathing is detected (output of S130 is “YES”) within the buffer period (i.e., follow on signals reveal a continued absence of breathing as judged by judging circuit 136), the controller 132 issues a control signal to emit an audible tone at alarm 133 (S135) and/or to energize the LED alarm 135.

If the judging circuit 136 determines that an apnea state has been reached (output of S140 is “YES”), the controller 132 of the main control unit 130 sends, via wireless/wired outputting circuit 138, controls signals to the vibration devices 120 via lead 127 9 or as a wireless signal) to initiate a tactile action (S160) in the gel mattress 110. The period of the absence of breathing is greater than the buffer period, and like the buffer period can be varied by control algorithms run by controller 132. As an example, for a neonatal or infant an apnea state may be determined once three (3) seconds have elapsed without any detection of breathing activity. For an adult, this period could be longer, such as 5 seconds for example.

In one example, upon determination of the apnea state the controller 132 directs one or more vibration devices 120 to begin vibrating so as to jog the patient to begin breathing again. The monitoring routines continue through the tactile action to detect the resumption of movement (S110) so as to terminate the tactile action once breathing is detected again.

In an example, given algorithm functions under controller 132 can generate specific control signals to be sent by the wireless/wired outputting circuit 138 so that the vibration devices 120 are energized in a particular sequence, such as starting at the feet and moving upward toward the chest and neck area of the patient. In another variant, the frequency or intensity of vibration can be increased (“progressive vibration”) the longer a patient does not begin to breathe again after the initial stimulation. In this example, the controller 132, based on data signals indicating continued absence of breathing, may selectively send different control signals such that the vibration intensity changes at the vibration devices. In a further variant, upon detection of the apnea state, the controller 132 (in addition to energizing the vibration devices 120) can issue another control signal to emit a second audible tone at alarm 133, which may be different in tone, pitch and/or volume than the alarm initiated after the buffer period has been exceeded. Should the apnea state persist, the tone of the second alarm can gradually change/increase, and/or the volume of the alarm can gradually increase, and/or the pitch of the alarm can change to alert a caregiver (and/or the sleeping person) of a continued apnea state condition.

The example system 100 enables monitoring of a sleeping person without requiring sensors to be physically attached to the body of the person. The gel mattress 110 provides for accurate monitoring of a person in any position and through any movement thereon. The immediate tactile stimulation provided by the example method and system once an apnea state condition is determined may quickly restore breathing of the sleeping person, independent of any caregiver action. The progressive vibration and/or variance in tone, pitch and/or volume of the alarm may facilitate the restoration of breathing and the alerting of the caregiver.

The example embodiments being thus described, it will be obvious that the embodiments may be varied in many ways. The example embodiments have been described where the stimulation devices are embodied as vibration devices 120 as a means of providing tactile stimulation to a person. However, other stimulation devices may be employed.

For example, instead of or in addition to having the vibration devices 120 therein, the gel mattress 110 may include a plurality of spaced, independent oscillating devices to provide a tactile action to the person. The oscillating devices are designed to produce a side-to-side or “wiggle” action to be felt by the person on gel mattress 110, distinct from the vibration devices. The oscillation devices may be off-the-shelf components such as are found in massage chairs, or may be smart devices with microcontrollers that can alter between a vibration and a wiggle based on type the control signal received from the wireless/wired outputting circuit 138, for example. Such variations are not to be regarded as departure from the example embodiments, and all such modifications as would be obvious to one skilled in the art are intended to be included herein.

What is claimed is:

1. A system for monitoring a sleeping person to detect an apnea state condition, comprising:
a pad provided beneath the person,
a plurality of independent position sensors arranged within the pad for measuring movement of the person at different locations,
a main control unit in electrical communication with the position sensors, and
a plurality of independent vibration devices arranged within the pad and in electrical communication with the main control unit,
wherein the main control unit is configured to receive signals from the position sensors indicative of a stoppage of breathing in the person for a given duration and to send control signals to cause the vibration devices to generate a tactile action in the pad so as to jog the person out of the apnea state.

2. The system of claim 1, wherein a delay or buffer period to detect no breathing is set within the control programming of the main control unit to avoid a spurious alarm.

3. The system of claim 1, wherein the main control unit further includes an audible alarm in addition to the tactile action that is triggered after no breathing is detected within a given period.

4. The system of claim 3, wherein the alarm changes in one of tone, pitch and volume should the apnea state condition persist.

5. The system of claim 1, wherein the pad is configured as a gel mattress containing the sensors and vibration devices therein.

6. The system of claim 1, wherein the main control unit further includes a display unit to display at least respiratory rate and heart rate thereon.

7. The system of claim 1, wherein the position sensors, main control unit and vibration devices communicate wirelessly or over a wired interface.

8. The system of claim 1, further comprising a plurality of independent oscillating devices arranged within the pad and in electrical communication with the main control unit, the main control unit, upon receiving signals from the position sensors indicative of a stoppage of breathing in the person for a given duration, configured to send control signals to cause the oscillating devices to generate a side-to-side action so as to jog the person out of the apnea state.

9. The system of claim 1, wherein the vibration devices receive control signals so as to be energized in a particular sequence on the pad.

10. The system of claim 1, wherein the vibration devices receive control signals to increase a frequency or severity of vibration the longer a patient does not begin to breathe after the initial tactile action.

11. A pad for monitoring a sleeping person to detect an apnea state condition, comprising:
a pair of sheets encasing a gel material therein for placement on a mattress or for use as a mattress,
a plurality of independent position sensors within the gel material for measuring movement of the person at different locations, and
a plurality of independent vibration devices within the gel material, the sensors and vibration devices in electrical communication with a remote main control unit,
wherein in response to the sensors sending signals to the main control unit indicative of a stoppage of breathing in the person for a given duration, the vibration devices receive control signals to generate a tactile action in the pad so as to jog the person out of the apnea state.

12. The pad of claim 11, wherein the position sensors, main control unit and vibration devices communicate over a wired interface or wirelessly.

13. The pad of claim 11, wherein the vibration devices receive control signals so as to be energized in a particular sequence on the pad.

14. The pad of claim 11, wherein the vibration devices receive control signals to increase a frequency or severity of vibration the longer a patient does not begin to breathe after the initial tactile action.

15. The pad of claim 11, further comprising a plurality of independent oscillating devices arranged within the gel material and in electrical communication with the main control unit, the main control unit, upon receiving signals from the position sensors indicative of a stoppage of breathing in the person for a given duration, being configured to send control signals to cause the oscillating devices to generate a side-to-side action so as to jog the person out of the apnea state.

16. A method of monitoring a sleeping human being to detect an apnea state condition, comprising:
providing a pad under a prone person prior to a sleep event, the pad having a plurality of independent position sensors therein for measuring movement of the person at different locations and a plurality of independent stimulation devices therein,
electrically connecting the plurality of position sensors and stimulation devices to a controller,
monitoring, by the sensors, movement of the person on the pads so as to track breathing, and
transmitting control signals to cause the stimulation devices to generate a tactile action in the pad so as to jog the person out of the apnea state condition, if the controller receives signals from the position sensors indicative of a stoppage of breathing in the person for a given duration.

17. The method of claim 16, wherein the position sensors, main control unit and stimulation devices communicate over a wired interface or wirelessly.

18. The method of claim 16, wherein transmitting control signals to the stimulation devices further includes transmitting the control signals to a plurality of vibration devices to energize the vibration devices in a particular sequence on the pad.

19. The method of claim 16, wherein transmitting control signals to the stimulation devices further includes transmitting the control signals to a plurality of vibration devices to increase the frequency or severity of vibration the longer a patient does not begin to breathe after the initial tactile action.

20. The method of claim 16, wherein transmitting control signals to the stimulation devices further includes transmitting the control signals to a plurality of independent oscillating devices arranged within the pad to cause the oscillating devices to generate a side-to-side action so as to jog the person out of the apnea state, if the controller receives signals from the position sensors indicative of the stoppage of breathing in the person for the given duration.

21. The method of claim 16, further comprising:
energizing an alarm upon determination of the apnea state condition, and
changing one of a tone, pitch and volume of the alarm should the apnea state condition persist.
